(19) United States
(12) Patent Application Publication
(10) Pub. No.: US 2025/0029822 A1
(43) Pub. Date: Jan. 23, 2025

(54) COMPOUND ASSEMBLY

(71) Applicants: Thermo Fisher Scientific (Bremen) GmbH, Bremen (DE); Thermo Finnigan LLC, San Jose, CA (US)

(72) Inventors: Martin Strohalm, Bremen (DE); Ralf Tautenhahn, Mountain View, CA (US)

(73) Assignees: Thermo Fisher Scientific (Bremen) GmbH, Bremen (DE); Thermo Finnigan LLC, San Jose, CA (US)

(21) Appl. No.: 18/771,302

(22) Filed: Jul. 12, 2024

Related U.S. Application Data
(60) Provisional application No. 63/528,253, filed on Jul. 21, 2023.

(51) Int. Cl. H01J 49/00

(52) U.S. Cl. CPC H01J 49/0036

(57) ABSTRACT

A method of processing mass spectral data is provided. The mass spectral data includes a plurality of MS¹ mass spectra and a plurality of MS^{N} mass spectra each having a respective associated retention time. A group of features is detected in the plurality of MS¹ mass spectra, each feature of the group having a respective mass, and the features of the group having corresponding retention times. The method includes, for each of one or more features of the group: submitting a corresponding MS^{N} mass spectrum to a mass spectral search engine in order to obtain an identification result for that feature, and determining a candidate ion type for the feature based on a mass difference between the mass associated with the feature and an expected mass from the identification result. The method also includes identifying one or more compounds based on the group of features and the candidate ion type(s).

### CROSS-REFERENCE TO RELATED APPLICATION

This application claims the benefit of U.S. Provisional Application No. 63/528,253, filed Jul. 21, 2023, entitled “COMPOUND ASSEMBLY,” which application is incorporated herein by reference in its entirety for all purposes.

### FIELD

The present invention relates to the field of mass spectrometry, and more particularly to methods of processing mass spectral data.

### BACKGROUND

It has been some time since the discovery of soft ionisation techniques enabled mass spectrometric analysis of intact small molecules, peptides and proteins. Using these techniques, very limited in-source fragmentation significantly simplifies the acquired spectra. However, single compounds still manifest in a series of mass to charge ratio (m/z) signals. Beside isotopes, these signals include solvent adducts, homo- or hetero-dimers, different charge states, and any remaining in-source fragments. The correct “assembly” of these multiple m/z signals is an important step in both qualitative and quantitative analysis.

It is believed that there remains scope for improvements to methods for processing mass spectral data.

### SUMMARY

A method of processing mass spectral data for a sample is provided. The mass spectral data comprises a plurality of MS¹ mass spectra and a plurality of MS^{N} mass spectra, with each mass spectrum of the plurality of MS¹ mass spectra and the plurality of MS^{N} mass spectra having a respective associated retention time. The method comprises detecting a group of features in the plurality of MS¹ mass spectra, wherein each feature of the group has a respective mass, and wherein the features of the group have corresponding retention times. The method comprises identifying one or more compounds in the sample based on the group of features.

As is described in more detail below, various embodiments provide improved methods of processing mass spectral data.

In embodiments, the mass spectral data is produced by an analytical instrument that comprises a chromatographic separation device (such as a liquid chromatography (LC) separation device or a gas chromatography (GC) separation device) and a mass spectrometer. The chromatographic separation device may separate the sample in a chromatographic separation scan, and the mass spectrometer may obtain the mass spectral data during the chromatographic separation scan. Thus, each mass spectrum of the plurality of MS¹ mass spectra and the plurality of MS^{N} mass spectra will have a respective associated retention time, i.e. each mass spectrum will have been obtained at a respective chromatographic retention time during the chromatographic separation scan.

As described further below, in some embodiments each MS^{N} mass spectrum is an MS² mass spectrum. However, each MS^{N} could instead be a higher order fragmentation spectrum such as an MS³ spectrum. In general, N is an integer ≥2.

The mass spectral data may be comprised of (i.e. may be formed from) at least one sample file, with each sample file being the data output from the mass spectrometer from a respective chromatographic separation scan. The mass spectral data of each sample file may comprise plural MS¹ mass spectra of the plurality of MS¹ mass spectra and plural MS^{N} mass spectra of the plurality of MS^{N} mass spectra. The mass spectral data may comprise a single such sample file, or a plurality of sample files. Where there are plural sample files, each sample file may be obtained from a chromatographic separation scan for a different fraction of the same sample.

The method comprises detecting a group of features in the plurality of MS¹ mass spectra. Each feature of the group may have a respective mass and a respective retention time associated with it. The features of the group may have corresponding (e.g. equal within a first tolerance) retention times, but different masses. The method may comprise detecting one or more further groups of features in the plurality of MS¹ mass spectra, with each respective different group having a respective different retention time. Although the processing of only a single group of features is described in detail below, it will be understood that each further group of features may be processed in a similar manner.

In some embodiments, each group of features is detected by firstly detecting features in the plurality of MS¹ mass spectra, and then grouping detected features that have corresponding (i.e. equal within the first tolerance) retention times. In turn, features may be detected in the plurality of MS¹ mass spectra by firstly detecting features-per-file in the MS¹ mass spectra of each sample file, and then grouping features-per-file across sample files that have corresponding masses and retention times to form features.

Thus, the step of detecting a group of features in the plurality of MS¹ mass spectra may comprise: for each sample file: detecting a plurality of features-per-file in that sample file, with each feature-per-file having a respective mass and a respective retention time; forming a plurality of features from the features-per-file, with each feature having a respective mass and a respective retention time; and forming a group of features by grouping features that have corresponding retention times.

In these embodiments, each feature-per-file in a sample file may be detected by firstly constructing a chromatogram for each unique mass-to-charge ratio (m/z) in the plural MS¹ mass spectra of the sample file and determining a characteristic retention time for each chromatogram. The characteristic retention time for a chromatogram may be the retention time at the centre or apex of the chromatogram, and may be determined, e.g., using a peak-detection algorithm or similar. The chromatograms may then be grouped into sets according to their characteristic retention times, and a de-isotoping algorithm may be applied to each set of chromatograms in order to form a group of features-per-file.

Thus, each feature-per-file is a feature appearing in the de-isotoped MS¹ mass spectral data that has a unique combination of mass and retention time. Then, each feature-per-file of each group of features-per-file will have a respective mass and a respective retention time, and the features-per-file of each group will have corresponding (i.e. the same within a second tolerance) retention times.

Equally, the step of detecting a plurality of features-per-file in a sample file may comprise: constructing a plurality of chromatograms from the plural MS¹ mass spectra of the sample file, with each chromatogram having a respective mass-to-charge ratio (m/z);
determining a characteristic retention time for each chromatogram; grouping chromatograms that have corresponding characteristic retention times into one or more sets of chromatograms; and applying a de-isotoping algorithm to each set of chromatograms to form a group of features-per-file.

As described above, features in each group of features may have retention times that are equal within a first tolerance, whereas features-per-file in each group of features-per-file may have retention times that are equal within a second tolerance. The second tolerance may be less than the first tolerance. As described further below, this has the effect of properly accounting for retention time differences between different sample files.

Thus, the step of grouping features that have corresponding retention times may comprise grouping features that have retention times that are equal within a first tolerance; the step of grouping chromatograms that have corresponding characteristic retention times may comprise grouping chromatograms that have retention times that are equal within a second tolerance; and the second tolerance may be less than the first tolerance.

In some embodiments, the method comprises: for each of one or more features of a group of features: (i) submitting a corresponding MS^{N} mass spectrum to a mass spectral search engine in order to obtain an identification result for that feature, and (ii) determining a candidate ion type (such as a candidate adduction type) for the feature based on any mass difference between the mass associated with the feature and an expected mass from the identification result. In this case, the step of identifying one or more compounds in the sample may be based both on the group of features and the candidate (adduct) ion type(s).

In these embodiments, the steps (i) and (ii) may be performed for those features of the group for which corresponding MS^{N} data is available in the mass spectral data. Where there are plural sample files, a corresponding MS^{N} mass spectrum may be taken from any of the sample files. Thus, an MS^{N} mass spectrum that corresponds to a feature may be an MS^{N} spectrum (from the plurality of MS^{N} mass spectra) for any one of the features-per-file corresponding to the feature.

As mentioned above, it would be possible for the mass spectral data to comprise only a single sample file, in which case each feature of the plurality of features is formed from one feature-per-file of the plurality of features-per-file.

In these embodiments, the step of identifying one or more compounds based on the group of features may comprise: (i) determining one or more clusters of features, wherein each cluster of features includes one or more features of the group, and possibly corresponds to a respective (single) compound; (ii) determining, for the group of features, one or more arrangements of the clusters of features, wherein each arrangement includes one or more non-conflicting clusters of features; and (iii) selecting, for the group of features, a preferred arrangement from the one or more arrangements of clusters of features; and then: identifying one or more compounds based on the preferred arrangement of clusters of features.

However, in particular embodiments, the mass spectral data comprises a plurality of sample files, and each feature of the plurality of features is formed by grouping features-per-file across the samples files that have corresponding masses and corresponding retention times. In this case, for each group of features there is a corresponding group of features-per-file in each sample file of the plurality of sample files.

Then, the step of identifying one or more compounds based on the group of features may comprise: for each group of features-per-file in a respective sample file: (i) determining one or more clusters of features-per-file, wherein each cluster of features-per-file includes one or more features-per-file of the group and possibly corresponds to a respective compound; (ii) determining, for the group of features-per-file, one or more arrangements of the clusters of features-per-file, wherein each arrangement includes one or more non-conflicting clusters of features-per-file; and (iii) selecting, for the group of features-per-file, a preferred arrangement from the one or more arrangements of clusters of features-per-file; and then, based on the preferred arrangements for the plurality of sample files: (iv) determining, for the group of features, one or more arrangements of clusters of features, wherein each cluster of features includes one or more features of the group of features and possibly corresponds to a respective compound, and wherein each arrangement includes one or more non-conflicting clusters of features; and (v) selecting, for the group of features, a preferred arrangement from the one or more arrangements of clusters of features; and then identifying one or more compounds based on the preferred arrangement of clusters of features.

As is described in more detail below, this two-step process of firstly determining a preferred arrangement in respect of each sample file, and then resolving any conflicts between the preferred arrangements from different sample files has the effect of increasing the reliability and consistency of compound identification when there are multiple sample files.

In some embodiments, the step of determining one or more clusters of features-per-file may comprise, for each group of features-per-file in a respective sample file: assigning one or more candidate ion types to each feature-per-file of the group; determining one or more candidate relationships between features-per-file of the group; and resolving any conflicts between the candidate ion types and the candidate relationships for the group.

In these embodiments, the step of assigning one or more candidate ion types to each feature-per-file of the group may comprise assigning one or more candidate ion types from plural different categories to each feature-per-file of the group. The candidate ion type categories may include, e.g., (i) identified ion types; (ii) user-defined base ion types; (iii) default ion types; and (iv) in-source fragment ion types.

Equally, the step of determining one or more candidate relationships (or “transitions”) between features-per-file of the group may comprise determining one or more candidate relationships from plural different categories of relationships. The categories of relationships may include, e.g., (i) in-source fragment relationships; and (ii) adduct relationships.

Thus, for example, each feature-per-file of the group may have a respective charge, and the step of assigning one or more candidate ion types to each feature-per-file of the group may comprise: assigning an identified ion type to any feature-per-file of the group that corresponds to a feature for which an identification result was obtained; and/or assigning a user-defined base ion type or a default ion type to each feature-per-file of the group based on the respective charge of the feature-per-file.

Additionally or alternatively, the step of assigning one or more candidate ion types to each feature-per-file of the group may comprise: assigning an in-source fragment ion type to any feature-per-file of the group that has a mass corresponding to the mass of an expected in-source fragment of another feature-per-file in the group.

In this case, the step of determining one or more candidate relationships between features-per-file of the group may comprise: determining an in-source fragment relationship between a feature-per-file of the group and another feature-per-file of the group when the feature-per-file has a mass corresponding to the mass of an expected in-source fragment of the other feature-per-file (or, equivalently, when the other feature-per-file has a mass corresponding to the mass of an expected in-source fragment of the feature-per-file).

In these embodiments, the mass of one or more expected in-source fragments of a feature-per-file may be determined from an MS^{N} mass spectrum (from the plurality of MS^{N} mass spectra) corresponding to that feature-per-file.

Alternatively, the method may comprise providing, as part of the identification result for a feature, the mass of one or more expected in-source fragments of that feature. In this case, the masses of the one or more expected in-source fragments of a feature-per-file may be derived from the provided mass(es).

Furthermore, the mass(es) provided as part of the identification result may be determined from one or more MS^{N} mass spectra configured to simulate in-source fragmentation. As is described in more detail below, by using custom MS^{N} mass spectra in this way, the identification of in-source fragmentation can be significantly improved.

In some embodiments, the step of determining one or more candidate relationships between features-per-file of the group may comprise determining one or more candidate adduct relationships between features-per-file of the group based on allowed mass shifts between the masses of the features-per-file of the group.

In embodiments, once all possible candidate ion types and candidate relationships have been determined for the group of features-per-file, any conflicts are resolved.

In some embodiments, the step of resolving any conflicts between the candidate ion types and the candidate relationships may comprise: removing any candidate relationships that conflict with a feature-per-file assigned as an identified ion type (e.g. removing any candidate adduct relationships and/or removing any candidate in-source fragment relationships that conflict with a feature-per-file assigned as an identified ion type); and/or removing any illegal candidate in-source fragment relationships.

As mentioned above, in some embodiments, the method comprises determining one or more clusters of features-per-file for each group of features-per-file in a respective sample file. Each such determined cluster may potentially correspond to a respective compound.

Thus, in embodiments, the step of determining one or more clusters of features-per-file may comprise: determining most or all possible clusters of features-per-file; and then removing any invalid cluster(s); and/or removing any cluster that does not include a feature-per-file assigned as a user-defined base ion.

As also mentioned above, the method may comprise determining one or more arrangements of clusters (of features-per-file or of features) for each group, and then selecting one of the arrangements as a preferred arrangement. Each such arrangement includes one or more clusters that do not conflict with one another. Any cluster that does not have a conflicting cluster can immediately be used in the preferred arrangement, without further processing.

Thus, the step of determining one or more arrangements of clusters (of features-per-file or of features) may comprise: determining if a cluster conflicts with one or more other clusters; and when it is determined that a cluster does not conflict with any other cluster, using that cluster in the preferred arrangement of clusters for the group.

On the contrary, where there are conflicting clusters, these must be resolved against one another. In embodiments, this is done by determining plural different arrangements of clusters, giving each arrangement a score, and selecting the arrangement with the highest score as the preferred arrangement.

Thus, the step of selecting a preferred arrangement of clusters (of features-per-file or of features) from the one or more arrangements of clusters may comprise: determining a score for each arrangement of clusters; and selecting the arrangement with the highest score.

The step of determining a score for each arrangement of clusters may comprise: determining a cluster score for each cluster in the arrangement by: (i) assigning a weight factor to each candidate ion type assignment of the cluster, (ii) assigning a relationship score to each candidate relationship of the cluster, and (iii) calculating a cluster score for the cluster by dividing the sum of the weight factors and relationship scores by the number of features or features-per-file in the cluster; and determining a score for each arrangement by: dividing the sum of the cluster scores for the arrangement by the number of clusters in the arrangement.

A further aspect provides a method of mass spectrometry comprising: analysing a sample to obtain mass spectral data that comprises a plurality of MS¹ mass spectra and a plurality of MS^{N} mass spectra, with each mass spectrum having a respective associated retention time; and processing the mass spectral data using the method(s) described above.

A further aspect provides a non-transitory computer readable storage medium storing computer software code which when executed on a processor performs the method(s) described above.

A further aspect provides a control system for an analytical instrument such as a mass spectrometer, the control system configured to cause the analytical instrument to perform the method(s) described above.

A further aspect provides an analytical instrument, such as a mass spectrometer, comprising the control system described above.

### BRIEF DESCRIPTION OF THE DRAWINGS

Various embodiments will now be described in more detail with reference to the accompanying Figures, in which:

FIG. 1 shows schematically a mass spectrometer that may be operated in accordance with embodiments;

FIG. 2 shows schematically a method according to embodiments;

FIG. 3 shows a representation of a main relationship graph comprising a plurality of features;

FIG. 4 shows a representation of a per-file relationship graph comprising a plurality of features-per-file;

FIG. 5A shows a representation of a feature-per-file having an identified ion type, FIG. 5B shows a representation of a feature-per-file that has been labelled as being either of two different user-defined base ion types, FIG. 5C shows a representation of a feature-per-file that has been labelled as being a default ion type, FIG. 5D shows a representation of a feature-per-file that has been labelled as being either of the two different user-defined base ion types or an in-source fragment ion;

FIG. 6 shows a representation of a per-file relationship graph comprising two features-per-file and including various possible ion types and transitions;

FIGS. 7A and 7B illustrate a process of removing invalid transitions from an initial per-file relationship graph;

FIG. 8 shows a representation of a per-file relationship graph comprising eight features-per-file and including various possible ion types and transitions;

FIG. 9 shows a representation of various possible clusters formed from the features-per-file of FIG. 8;

FIG. 10 illustrates a process of removing invalid clusters from an initial set of possible clusters;

FIG. 11 illustrates a process of removing invalid orphan clusters from an initial set of possible clusters;

FIG. 12 shows a representation of various possible clusters formed from the features-per-file of FIG. 8 after invalid clusters have been removed;

FIG. 13 illustrates a process of determining various possible explanations for the group of features-per-file from the various possible clusters of FIG. 12;

FIG. 14 illustrates a final explanation for the group of features-per-file as determined in accordance with embodiments;

FIG. 15 illustrates a process of resolving fragments; and

FIG. 16A illustrates experimental results using a conventional assembly method, and FIG. 16B illustrates experimental results using the assembly method of various embodiments.

### DETAILED DESCRIPTION

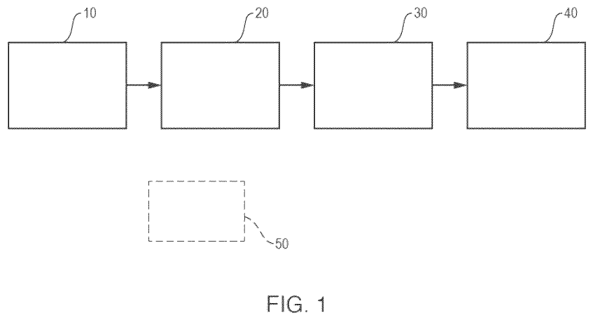

FIG. 1 illustrates schematically an analytical instrument, such as a mass spectrometer, that may be used in conjunction with the methods described herein. As shown in FIG. 1, the instrument includes an ion source 10, a mass filter 20, a fragmentation device 30, and a mass analyser 40.

The ion source 10 is configured to generate ions from a sample. The ion source 10 may be coupled to a chromatographic separation device (not shown) such as a liquid chromatography (LC) separation device, a gas chromatography (GC) separation device, or a capillary electrophoresis separation device, such that the sample which is ionised in the ion source 10 comes from the separation device. The ion source 10 can be any suitable ion source, such as an electrospray ionisation (ESI) ion source, an atmospheric pressure ionisation (API) ion source, a chemical ionisation ion source, an electron impact (EI) ion source, or similar.

The mass filter 20 is arranged downstream of the ion source 10, and is configured to receive ions from the ion source 10. The mass filter 20 is configured to filter the received ions according to their mass to charge ratio (m/z). The mass filter 20 may be configured such that received ions having m/z within an m/z transmission window of the mass filter are onwardly transmitted by the mass filter, while received ions having m/z outside the m/z transmission window are attenuated by the mass filter, i.e. are not onwardly transmitted by the mass filter. The width and/or the centre m/z of the transmission window may be controllable (variable), e.g. by suitable control of RF and/or DC voltage(s) applied to electrodes of the mass filter 20. Thus, for example, the mass filter 20 may be operable in a transmission mode of operation, whereby most or all ions within a relatively wide m/z window are onwardly transmitted by the mass filter 20, and a filtering mode of operation, whereby only ions within a relatively narrow m/z window (centred at a desired m/z) are onwardly transmitted by the mass filter 20. The mass filter 20 can be any suitable type of mass filter, such as a quadrupole mass filter.

The fragmentation device 30 is arranged downstream of the mass filter 20, and is configured to receive most or all ions transmitted by the mass filter 20. The fragmentation device 30 may be configured to selectively fragment some or all of the received ions, i.e. so as to produce fragment ions. The fragmentation device 30 may be operable in a fragmentation mode of operation, whereby most or all received ions are fragmented so as to produce fragment ions (which may then be onwardly transmitted from the fragmentation device 30), and a non-fragmentation mode of operation, whereby most or all received ions are onwardly transmitted without being (deliberately) fragmented. It would also be possible for a non-fragmentation mode of operation to be implemented by causing ions to bypass the fragmentation device 30. The fragmentation device 30 may also be operable in one or more intermediate modes of operation, e.g. whereby the degree of fragmentation is controllable (variable). The fragmentation device 30 can also be operable in higher order (MS^{N}) fragmentation modes of operation, e.g. whereby fragment ions are further fragmented one or more times by the fragmentation device 30.

The fragmentation device 30 can be any suitable type of fragmentation device, such as for example a collision induced dissociation (CID) fragmentation device, an electron induced dissociation (EID) fragmentation device, a photodissociation fragmentation device, and so on. Numerous other types of fragmentation are possible.

In some embodiments, the fragmentation device 30 is a collision induced dissociation (CID) fragmentation device. Thus, the fragmentation device may include a collision cell which may be filled with a collision gas, e.g. maintained at a relatively high pressure. Ions may be selectively fragmented in the collision cell by controlling (varying) the kinetic energy with which ions are caused to enter the collision cell. In a fragmentation mode of operation, ions may be accelerated so that they enter the collision cell with a relatively high kinetic energy, which may cause most or all of the accelerated ions to fragment. In a non-fragmentation mode of operation, ions may be caused to enter the collision cell with a relatively low kinetic energy, which may be insufficient to cause most or all of the ions to fragment. In intermediate modes, ions may be caused to enter the collision cell with intermediate kinetic energies.

The mass analyser 40 is arranged downstream of the fragmentation device 30 and is configured to receive ions from the fragmentation device 30. Thus, the mass analyser 40 may receive unfragmented precursor ions or fragment ions, depending on the mode of operation of the fragmentation device 30. The mass analyser 40 is configured to analyse the received ions so as to determine their mass to charge ratio (m/z) and/or mass, i.e. to produce a mass spectrum of the ions. The mass analyser 40 can be any suitable type of mass analyser, such as an ion trap mass analyser, an electrostatic orbital trap mass analyser (such as an Orbitrap™ FT mass analyser as made by Thermo Fisher Scientific) or a time-of-flight (ToF) mass analyser such as a multi-reflecting time-of-flight (MR-ToF) mass analyser.

It should be noted that FIG. 1 is merely schematic, and that the instrument can, and in embodiments does, include any number of one or more additional components. For example, the instrument may include one or more ion transfer stage(s) arranged between any of the illustrated components, e.g. including an atmospheric pressure interface and/or one or more ion guides, lenses and/or other ion optical devices configured such that some or all of the ions can be transmitted appropriately through the instrument. The ion transfer stage(s) may include any suitable number and configuration of ion optical devices, for example optionally including one or more ion guides, lenses and/or other ion optical devices.

In some embodiments, the instrument may include more than one mass analyser. For example, the instrument may be a dual mass analyser hybrid mass spectrometer of the type described in EP 3,410,463, the contents of which are incorporated herein by reference.

As also shown in FIG. 1, the instrument is under the control of a control unit 50, such as an appropriately programmed computer, which controls the operation of various components of the instrument and, for example, sets the voltages to be applied to the various components of the instrument. The control unit 50 may also receive and process data from various components including the analyser(s) in the manner of various embodiments.

The instrument may be operable in various mode of operation. For example, the instrument may be a tandem mass spectrometer operable in an MS1 mode of operation and an MS2 mode of operation.

In the MS1 (or “full mass scan”) mode of operation, the mass filter 20 is operated in its transmission mode of operation and the fragmentation device 30 is operated in its non-fragmentation mode of operation, e.g. so that a wide m/z range (e.g. full mass range) of unfragmented (“precursor” or “parent”) ions are analysed by the analyser 40 to produce an MS1 spectrum.

In the MS2 mode of operation, the mass filter 20 is operated in its filtering mode of operation and the fragmentation device 30 is operated in its fragmentation mode of operation, e.g. so that a selected narrow m/z range of precursor ions are fragmented and the resulting fragment (“product” or “daughter”) ions are analysed by the analyser 40 to produce an MS2 spectrum.

The instrument may also be operable in one or more higher order fragmentation modes of operation, such as for example an MS3 mode of operation, whereby precursor ions are fragmented, at least some of the resulting fragment ions are themselves fragmented, and the second-generation fragment ions (“granddaughter ions”) are analysed by the analyser 40 produce an MS3 spectrum. In general, the instrument may be operable in any order of fragmentation mode of operation, i.e. in an MS^{N} mode of operation where N≥2.

A method of operating the analytical instrument involves providing a sample to the chromatographic (e.g. LC or GC) separation device so that the sample is chromatographically separated, ionising the eluent from the chromatographic separation device in the ion source 10, and analysing the resulting ions. Different compounds within the sample experience different retention times (RT) within the chromatographic separation device and so elute from the chromatographic separation device (and are ionised) at different times. The chromatographic separation device typically takes a few tens of seconds or a few minutes to complete each chromatographic separation scan.

During each chromatographic separation scan, multiple MS2 spectra (or, more generally, multiple MS^{N} spectra) may be acquired by sequentially altering the centre of the mass filter's (narrow) m/z window between each of a plurality of different m/z values, e.g. so as to sequentially select (and fragment) each of a plurality of different precursor ions with respective different m/z.

In a data dependent acquisition (DDA) mode of operation, the plurality of different m/z values may correspond to a plurality of different precursor ions identified from corresponding MS1 data (i.e. a full mass scan). Thus, a typical data dependent acquisition (DDA) method involves repeatedly performing, during a chromatographic separation scan, the steps of: (i) obtaining an MS1 spectrum across an m/z range of interest; (ii) identifying one or more precursor ions of interest in the MS1 spectrum; and (iii) obtaining an MS2 (or MS^{N}) spectrum in respect of each the identified precursor ions of interest. Step (iii) comprises, for each of the identified precursor ions: isolating the precursor ion using the mass filter 20, fragmenting the isolated precursor ions in the fragmentation device 30, and mass analysing the fragment ions using the mass analyser 40.

In a data independent acquisition (DIA) MS2 (or MS^{N}) mode of operation, the plurality of different m/z values may be taken from a predetermined (fixed) list, i.e. without reference to MS1 data. For example, a narrow m/z isolation window may be sequentially stepped across the entire m/z range of interest, e.g. as described in EP 3,410,463.

In any case, each chromatographic separation scan performed by the analytical instrument will produce a sample file. Each sample file includes data in respect of multiple MS1 spectra, each with an associated retention time; and data in respect of multiple MS2 (or MS^{N}) spectra, each with an associated retention time and an associated mass filter isolation window or precursor ion m/z. A prepared sample may be fractionated and a chromatographic separation scan may be performed for each fraction to produce multiple such sample files in respect of the same sample.

The repetition rate of the DDA/DIA method may be fast enough to sample each chromatographically separated compound of interest multiple times during its chromatographic elution. So, for each sample file, a chromatogram trace may be constructed for each unique m/z of interest from the multiple MS1 spectra. Such chromatograms typically manifest as a peak corresponding to the chromatographic elution peak of each chromatographically separated compound.

The centre (e.g. apex) retention time (RT) of each chromatogram may then be determined, e.g. using a suitable peak detection algorithm or similar. Chromatograms with the same (e.g. within a certain tolerance) centre retention time (RT) may be grouped together, resulting in one or more sets of multiple m/zs, where all m/zs within a set have been determined to have the same retention time (RT).

Each such set of multiple m/zs can result from multiple different compounds that co-elute from the chromatographic separation device, and can be very complex and difficult to interpret. As such, there is a need to “assemble” each set of multiple m/zs into one or more clusters, where each cluster is a sub-set of m/zs from the set (or, where appropriate, the complete set) of multiple m/zs determined to have the same RT, with all m/zs in a cluster belonging to the same single compound. In other words, each single chromatographically separated compound can give rise to a cluster of multiple m/zs in MS1 data (having the same RT), giving rise to complexity in the MS1 data when there are multiple co-eluting compounds.

A single compound can give rise to multiple different m/zs in MS1 (at the same RT) due to, for example, different charge states (2) and different isotopes. A set of isotopes appears in MS1 data as a characteristic series of peaks separated from one another by 1 m/z for singly charged species, ½ m/z for doubly charged species, ⅓ for triply charged species, and so on. This understanding means that the MS1 data can be “de-isotoped”, e.g. by grouping those m/zs (in a set of multiple m/zs determined to have the same RT) that correspond to a set of isotopes together into a so-called “feature-per-file”, with each feature-per-file having a single characteristic m/z (e.g. corresponding to the lightest isotope). This understanding also means that the correct charge state can be determined for each such feature-per-file (from the m/z separation between isotopic peaks). (Any “singlets” appearing in the MS1 data (i.e. peaks that appear without any corresponding isotopes-typically low abundance peaks, where the corresponding isotopes are below the detection limit) can be assumed to be singly charged (or to have some other default charge, depending on the sample type etc.)) Knowledge of the charge state (z) and the mass to charge ratio (m/z) in turn means that a single characteristic mass (m) can be determined for each feature-per-file. Algorithms for such de-isotoping and charge state determination are known in the art.

However, despite such de-isotoping algorithms, multiple features-per-file can still be present for each compound. In other words, once each set of multiple m/zs has been de-isotoped, a group of multiple features-per-file may remain (where each such group of multiple features-per-file can result from a single compound or from multiple different co-eluting compounds), and so there is a need to further assemble each group of multiple features-per-file into one or more clusters, with all features-per-file in a cluster belonging to the same single compound.

The presence of multiple features-per-file for a single compound may have several different causes. In particular, this may be due to:
(i) Solvent adducts; i.e. adducting during the ionisation process may result in peaks in MS1 with a characteristic mass difference relative to the neutral mass M of a compound. For example, while the protonated ion [M+H]⁺ has a mass difference of ˜1, the ammonia adduct [M+NH4]⁺ has a mass difference ˜18, and so on.
(ii) Homo- or hetero-dimers; i.e. two proteins joined together. These may again manifest as characteristic mass differences in MS1.
(iii) In-source fragments; i.e. unintentional fragmentation of ions can lead to fragment ions appearing in MS1 spectra (not just MS2 spectra as desired), leading to difficultly in interpreting MS1 spectra.

Equally, where there are multiple sample files for the same sample, multiple “features” can exist for each compound (where a feature is the set of plural corresponding features-per-file from the multiple sample files). Ideally, all corresponding features-per-file of a feature would be identical in terms of m/z and RT, but in practice RT can differ slightly between different chromatographic separation runs. As such, there is a need to assemble each group of multiple features into one or more clusters, with all features in a cluster belonging to the same single compound.

Various embodiments are concerned with methods for assembling such features or features-per-file into clusters, i.e. determining which features or features-per-file relate to the same compound.

Known methods of performing such compound assembly are based solely on expected mass shifts between features-per-file. However, the inventors have now recognised that these approaches have several problems:
(i)(a) Compounds with intrinsic charge cannot be correctly identified. Some compounds exist with intrinsic charge and these typically do not form additional adduct ions. Therefore, there is no observable mass shift (between at least two different ions) in MS1, which would provide a hint for their correct assignment (i.e. it can't be determined from MS1 data alone whether an ion is, e.g., a protonated ion [M+H]⁺ or an ammonia adduct ion [M+NH4]⁺, etc., which needs to be known to be able to reliably determine the neutral mass M of the compound). In the known approach, these “orphan ions” may be incorrectly assigned, e.g. using a default ion definition (e.g. the protonated ion [M+H]⁺ for electrospray ionisation). As is described further below, various embodiments address this problem by performing a preliminary identification of the compound by searching its fragmentation (e.g. MS2 or MS^{N}) data against a database of standards (such as, e.g., the mzCloud™ database).
(i)(b) Relatedly, compounds not forming (de) protonated ions are often incorrectly identified. Some compounds preferably create one type of ion, which may be different from the default ion (e.g. different from the protonated ion [M+H]⁺ for electrospray ionisation). For example, some lipids tend to form sodium adduct ions [M+Na]⁺ only. This creates the same problem as above. As is described further below, various embodiments again address this problem by performing a preliminary identification of the compound by searching its fragmentation data against a database of standards.
(ii) Compound-specific in-source fragments are not identified. Many compounds undergo a small amount of fragmentation during ionisation (or elsewhere in the instrument during an MS1 scan) and so their fragments appear in MS1 spectra. These fragment ions may then be erroneously treated by the assembly algorithm as additional MS1 features, creating false positive hits. One example of this is PEG (polyethylene glycol) losing one or more units of EG (ethylene glycol). When analysed, it can appear as though the sample contains both PEGn6 and PEGn5, although the latter is actually a fragment created inside the instrument. As described below, various embodiments address this problem by obtaining reference MS2 (or MS^{N}) data for each feature (e.g. from the MS2 or MS^{N} data in the sample file(s) or by having the identification step (e.g. mzCloud™) return a standard MS2 or MS^{N} spectrum from a collection of low-energy MS2 or MS^{N} spectra (which may be configured to simulate in-source fragmentation)), and then searching the MS1 data for fragments that match the fragments in the reference MS2 or MS^{N} data.
(iii) Inconsistent adduct type assignment across multiple sample files. There are several reasons why different fractions of the same sample can produce different results in respect of adduct type assignment. This may be due, for example, to intensity/abundance variations between fractions leading to one or more of the adducts being below the detection limit, resulting in the whole adduct cluster being assigned differently. As is described below, embodiments address this problem by placing multiple, potentially competing, individual fraction explanations (or “arrangements”) into a final graph, which is resolved at the end of the process.

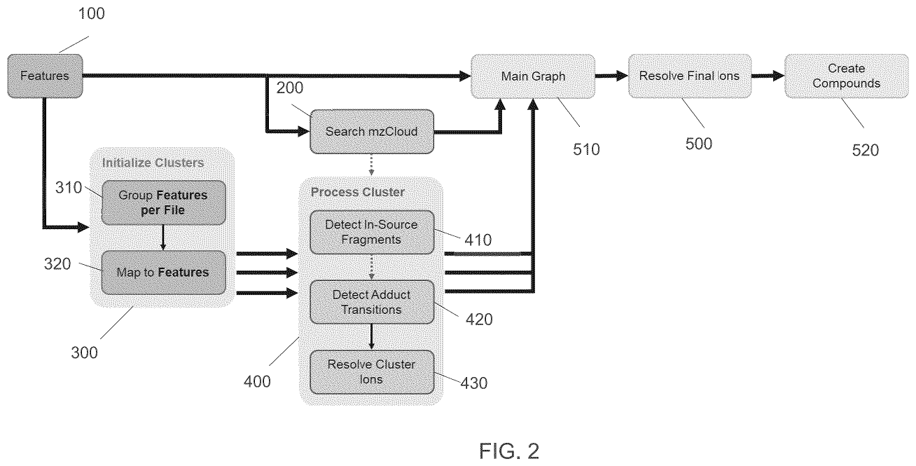

FIG. 2 illustrates a method in accordance with embodiments. As shown in FIG. 2, in a first step (step 100), features-per-file are detected in individual sample files (in the manner described above), and these are then grouped across the multiple sample files into features by matching their RT and m/z values. These consolidated features are inserted into a main relation graph 510, e.g. as nodes.

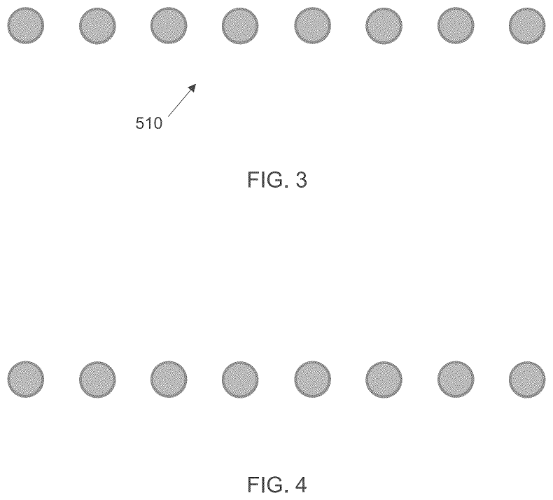

FIG. 3 shows one example illustration of such a main relation graph 510. In FIG. 3, each shaded circle (node) represents an identified feature.

Returning to FIG. 2, in step 200, if MS2 (or MS^{N}) data is available for any of the detected features (from the MS2 or MS data within any of the sample files for the same sample), this data is submitted to a database search algorithm for identification. Any suitable database search algorithm can be used, such as for example, the mzCloud™ spectral library. From the returned results (which will include the precursor ion neutral mass M), the possible adduct type assignment(s) are determined. In particular, mass shifts between the measured mass and the retrieved identification mass may be used to calculate the ion type of the feature. For example, if the mass measured in MS1 is (M+18), then the feature is identified as an ammonia adduct [M+NH4]⁺, and this information is inserted into the main relation graph 510 as one possible identification of the ion type.

This process not only allows high-confidence assignment of the ion type of a feature, but also provides a method to correctly identify compounds with intrinsic charge. Possible ion types may be taken directly from identification metadata or from one or more predefined list(s), or may be generated automatically for given charge state, e.g. by (de) protonation, if necessary.

Thus, in embodiments, a library search 200 is performed before final compound assembly at step 500. The results of the search 200 are not treated as being a definitive identification of the feature (as is conventional), but rather as one strong possibility which must later be resolved against any other competing explanations and identifications (e.g. from other sample files).

Steps 300 and 400 of FIG. 2 are carried out to group the features-per-file for each group of features-per-file (at the same RT) into possible clusters of related features-per-file. To address problem (iii) above, this process works on a per-file basis, then all the possible explanations are added to the main graph 510, and any conflicts between explanations from different sample files are resolved in step 500 (which will be described further below).

In step 300, the features-per-file in each sample file are grouped at step 310 by matching RT values (e.g. in the manner described above) and optionally one or more other properties like peak shape, etc. Because this process works on a per-file basis at this stage, a very narrow tolerance can be used to group features-per-file by RT. As shown, mapping to features is performed at step 320.

FIG. 4 shows one example illustration of a resulting per-file relation graph. In this per-file context, each shaded circle in FIG. 4 represents a feature-per-file in a group of features-per-file determined to have the same RT.

In step 400 of FIG. 2, the grouped features-per-file are analysed to determine all possible relationships between the various features-per-file in each group.

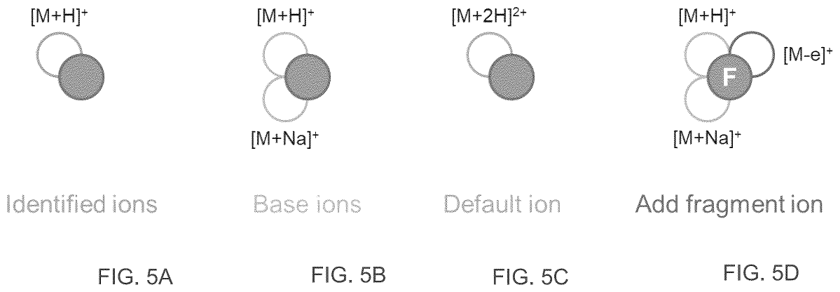

This involves firstly provisionally assigning all possible ion types to each feature-per-file of the group. Various possible categories of ion types are illustrated by FIGS. 5A-D, including identified ions (FIG. 5A), base ions (FIG. 5B), default ions (FIG. 5C), and fragment ions (FIG. 5D). At this stage, each feature-per-file can have multiple possible provisional ion type assignments, which are to be resolved later in the process.

Ion type identification information (from step 200) is added to those features-per-file for which the corresponding feature was identified in step 200. Thus, for example, FIG. 5A is representative of a feature-per-file of a feature that was identified as being a protonated ion [M+H]⁺ in step 200.

Each feature-per-file is also provisionally assigned as being one or more user-defined base ion(s) (FIG. 5B) depending on the known charge of the feature-per-file (where the charge of each feature-per-file is known from the de-isotoping algorithm, as described above (or from some other pre-processing of the data)), or else as a default ion (FIG. 5C) where the user did not define a base ion for ions with the known charge of that feature-per-file. The user-defined base ion(s) can be set as desired by the user, e.g. depending on the sample chemistry and/or configuration of the ion source, etc. As is described further below, in some embodiments, at least one user-defined base ion may be required to be present in a cluster for that cluster to be considered valid. The default ion may be the most common ion formed (e.g. by (de) protonation) during ionisation for ions with the charge of the feature-per-file. For example, for electrospray ionisation, the default ion may be set as a protonated ion [M+H]⁺ for singly positively charged ions, [M+2H]²⁺ for doubly positively charged ions, and so on.

Thus, for example, FIG. 5B is representative of a singly positively charged feature-per-file that has been provisionally assigned as being either of two user-defined base ions, namely either a protonated ion [M+H]⁺ or a sodium adduct ion [M+Na]⁺. FIG. 5C is representative of a doubly positively charged feature-per-file that has been provisionally assigned as being the default doubly positively charged ion, namely [M+2H]²⁺.

Returning again to FIG. 2, in step 410, the features-per-file in the group are analysed to determine whether any of the features-per-file could be an in-source fragment. For those features-per-file of the group for which MS2 (or MS^{N}) data is available for the corresponding feature (i.e. the same features that were identified in step 200), representative MS2 (or MS^{N}) data is obtained.

The representative MS2 (or MS^{N}) data may be the corresponding MS2 (or MS^{N}) data from the sample file(s), or more helpfully may be a standard MS2 (or MS^{N}) spectrum from a library of low-energy collision spectra. Such a spectrum may be returned together with the identification (e.g., from mzCloud™), and may be configured to accurately simulate in-source fragmentation. Thus, in embodiments, a new functionality is added to the search engine (e.g. to mzCloud™), which returns masses from low-energy collision spectra (e.g. HCD 10) as part of each identification hit.

Where the identification 200 was not successful and/or where low-energy collision data are not available, acquired MS2 (or MS^{N}) spectra from the raw file can instead be used as the representative MS2 (or MS^{N}). However, the assignment confidence is higher when the curated fragmentation data is used, which is based on authentic standards.

The representative MS2 (or MS^{N}) data is then compared to the other features-per-file in the group to determine whether any of the other features-per-file might be an in-source fragment of the feature-per-file under consideration, e.g. by looking for features-per-file in the group that have a mass that matches the mass of an in-source fragment in the representative MS2 (or MS^{N}) data.

Any of the features-per-file in the group that have such a matching mass are labelled as potentially being an in-source fragment. Thus, for example, FIG. 5D is representative of a singly positively charged feature-per-file that has been provisionally assigned as being either of the two user-defined base ions ([M+H]⁺ or a [M+Na]⁺) or an in-source fragment ion. As the ion type of an in-source fragment is generally unknown, a generic ion type assignment (e.g. [M-e]⁺) may be used.

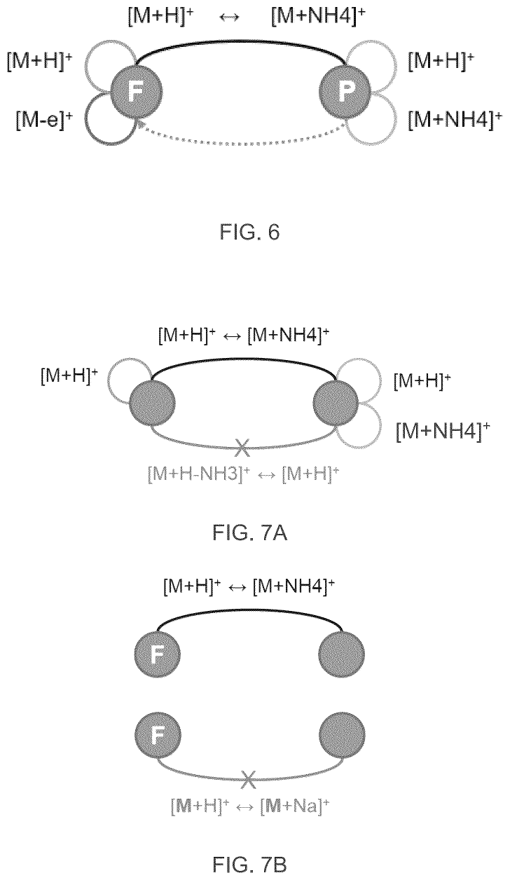

For possible in-source fragments, the relationship between the possible in-source fragment and the parent ion of that in-source fragment is also recorded in the graph. Thus, for example, in FIG. 6 this potential relationship is indicated by a dotted line connection between the nodes (with the feature-per-file labelled “P” potentially being the precursor ion of the in-source fragment “F”).

Returning once again to FIG. 2, in a next step (step 420), possible adduct relationships between the features-per-file in the group are determined by looking for allowed mass shifts between the masses of the features-per-file. A list of predefined ion types (e.g. adducts, common neutral losses, simple multimers, and charge states) is used to generate expected mass shifts, these expected mass shifts are applied to each group to find all possible relationships between the features-per-file of the group. Thus, for example, where two features-per-file have a mass difference of 17(=18−1), those two features could be a protonated ion [M+H]⁺ and its ammonia adduct [M+NH4]⁺; where the difference is 22(=23−1) the features-per-file could be [M+H]⁺ and [M+Na]⁺, and so on.

Any such possible relationships are recorded in the graph. Thus, for example, in FIG. 6 a potential adduct relationship is indicated by a solid line connection between the nodes, where the line is labelled according to the possible adduct relationship (i.e. [M+H]⁺↔[M+NH4]⁺ in this example).

FIG. 6 shows a simple example of a group of two features-per-file with various possible relationships labelled. In particular, the first feature (on the left) has been labelled as being either an identified ion of type [M+H]⁺ or an in-source fragment [M-e]⁺. The second feature (on the right) has been labelled as being either of two different base ions, namely either [M+H]⁺ or [M+NH4]⁺. In addition, in FIG. 6, the solid line labelled [M+H]⁺↔[M+NH4]⁺ is representative of a possible adduct transition, while the dotted line represents a possible in-source fragment relationship (with the feature labelled “P” being the precursor ion of the in-source fragment “F”).

As can be seen from the example of FIG. 6, the above-described process can result in multiple possible, potentially conflicting, relationships between the features-per-file in a group. Next, in step 430 of FIG. 2, these conflicts are resolved.

To do this, firstly, any conflicts between the ion-type assignments (loops) and the transitions (connections between nodes) are resolved. This process is illustrated by FIG. 7 and involves (i) removing any transitions that conflict with identified ions (FIG. 7A); and (ii) removing illegal fragment transitions (FIG. 7B).

FIG. 7A shows again the example of FIG. 6, but with an additional solid line labelled [M+H—NH3]⁺↔[M+H]⁺ that represents a second possible adduct transition (in addition to the transition [M+H]⁺↔[M+NH4]⁺). As can be seen in FIG. 7A, the in-source fragment label [M-e]⁺ for the first feature-per-file has been removed because this conflicts with the identified ion type [M+H]⁺ for the first feature-per-file. Then, the second possible adduct transition ([M+H—NH3]⁺↔[M+H]⁺) is removed because the [M+H—NH3]⁺ required for that transition also conflicts with the identified ion type [M+H]⁺ for the first feature-per-file.

FIG. 7B shows an example in which an illegal fragment transition is removed. Specifically, the fragment transition [M+H]⁺↔[M+Na]⁺ is removed because a [M+H]⁺ fragment cannot be created from a [M+Na]⁺ precursor (i.e. fragment transitions must not “create” new elements). In contrast, the possible fragment transition [M+H]⁺↔[M+NH4]⁺ is retained because the [M+H]⁺ fragment can be created from a [M+NH4]⁺ precursor.

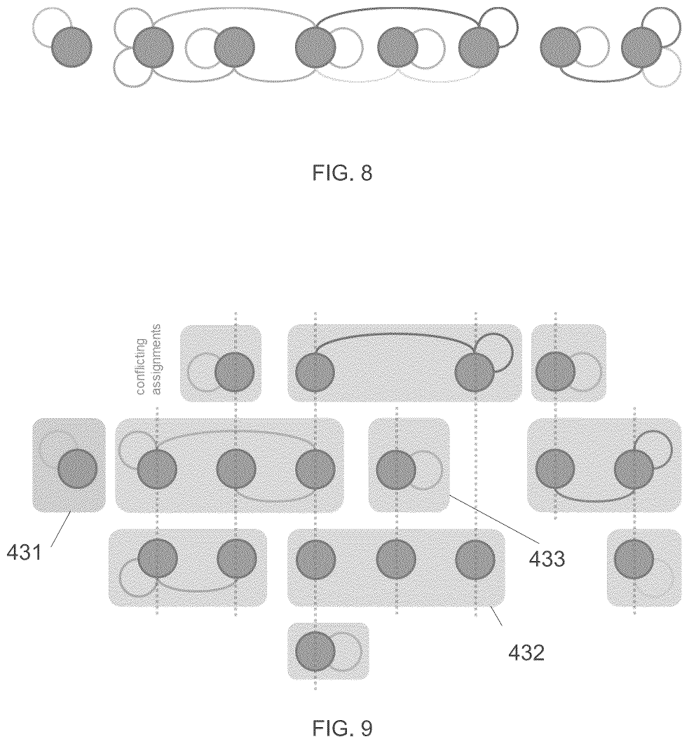

FIG. 8 shows a simplified example per-file graph that may result from the above-described process, with all remaining possible ion type assignments and transitions shown by the various loops and connections between the nodes. Several different explanations to assemble the features-per-file are still possible. From the graph of FIG. 8, all possible clusters (with each cluster corresponding to a single compound M) may be determined.

FIG. 9 shows the results of this determination. In FIG. 9, each possible cluster is shown as a shaded box. The left-most box 431 represents a cluster that has no other competing explanation (i.e. whose features-per-file do not appear in any other possible cluster). As such, this represents a successful identification of a cluster that need not be processed further (at the per-file level). This cluster 431 is added to the main graph 510 (to be resolved against any other competing explanation from other sample files later in step 500).

Each remaining cluster is a possible cluster that conflicts with a least one other possible cluster. These various conflicts are illustrated by the vertical dashed lines in FIG. 9. Next, these conflicts are resolved.

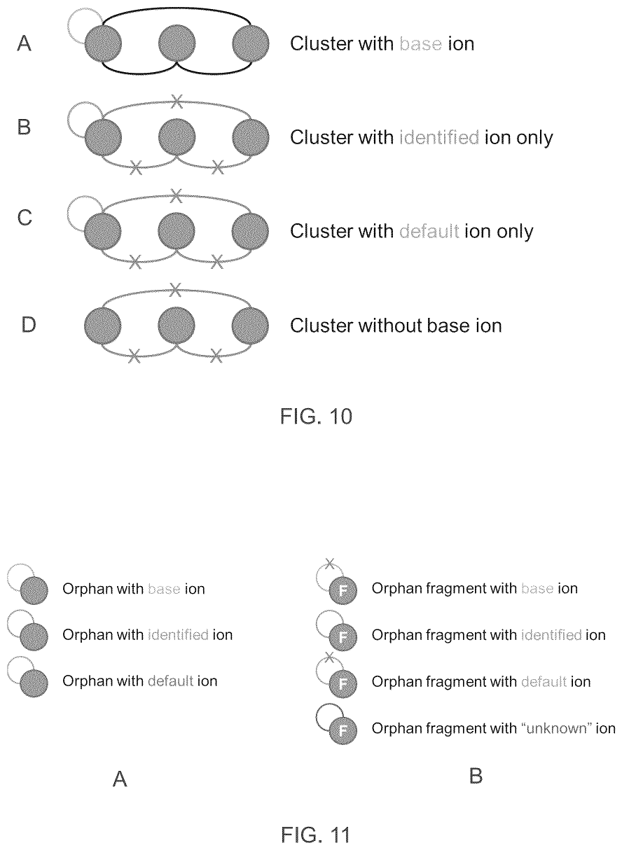

As illustrated by FIG. 10, in embodiments, where the user has provided one or more base ion types (as described above), any of the possible clusters that do not include one or more features-per-file assigned as potentially being one or more of the base ion types are rejected. (Where the user did not provide any required based ion type, this step may be skipped.) Thus, as illustrated by FIG. 10A, a cluster with a base ion may be retained. As illustrated by FIG. 10B, a cluster with only an identified ion may be rejected. As illustrated by FIG. 10C, a cluster with only a default ion may be rejected. As illustrated by FIG. 10D, a cluster without any base ion may be rejected.

Referring again to FIG. 9, in the illustrated example, the possible cluster labelled 432 is removed because there are just two transitions found, without any possible assignment (loops) of a base adduct ion or an identified adduct ion to any of the nodes within the cluster 432. The cluster 432 is therefore considered as invalid and is removed from further consideration.

Next, as illustrated by FIG. 11, after rejecting any invalid ion clusters, any orphan clusters (i.e. single features-per-file with no transitions to any other feature-per-file) that remain are considered. As illustrated by FIG. 11A, the assignment for any orphan clusters that have a valid ion type assignment (namely, base ion, identified ion, or default ion assignments) are retained. As illustrated by FIG. 11B, for orphan clusters labelled as potentially being an in-source fragment, the ion type assignment is retained only in the case of identified ions and generic fragment ions (whereas the ion type assignment is removed in the case of base ions and default ions).

Thus, in embodiments, step 430 comprises (i) removing ion type assignments with non-matching charge; (ii) removing ion type assignments that conflict with any pre-identified ion type; (iii) removing any clusters that are missing one or more user-specified base ion types; and (iv) removing any invalid fragment-precursor relationships (e.g. where additional atoms would need to be added to the fragment).

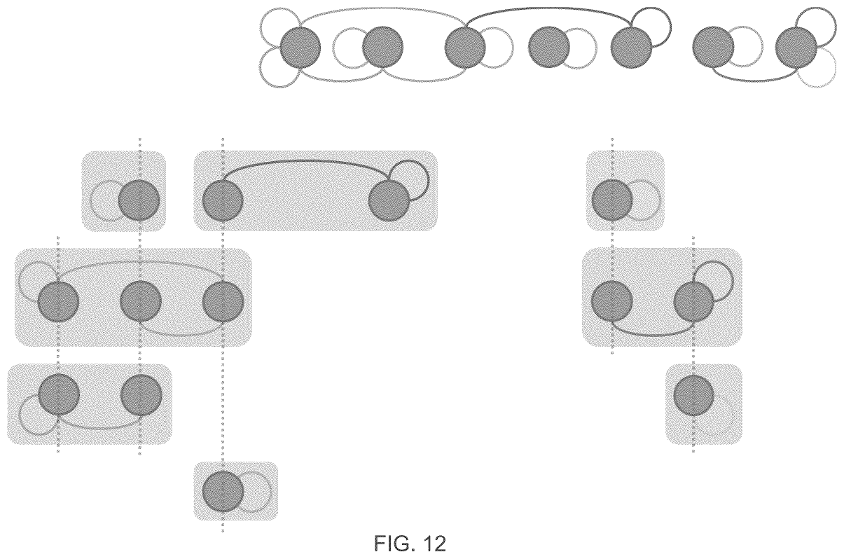

FIG. 12 shows the resulting graph after processing the graph of FIG. 9. As can be seen in FIG. 12, the clusters 431 and 432 from FIG. 9 have been removed (for the reasons given above). Furthermore, after removing the cluster 432, the orphan node 433 from FIG. 9 no longer conflicts with any other node, and so it is considered as being resolved, and has also been removed from the graph of FIG. 12.

As can be seen in FIG. 12, at this stage, there may still be multiple conflicting possible clusters. However, the graph has been resolved into two disconnected sub-graphs, which can each be further processed separately because they do not conflict with one another.

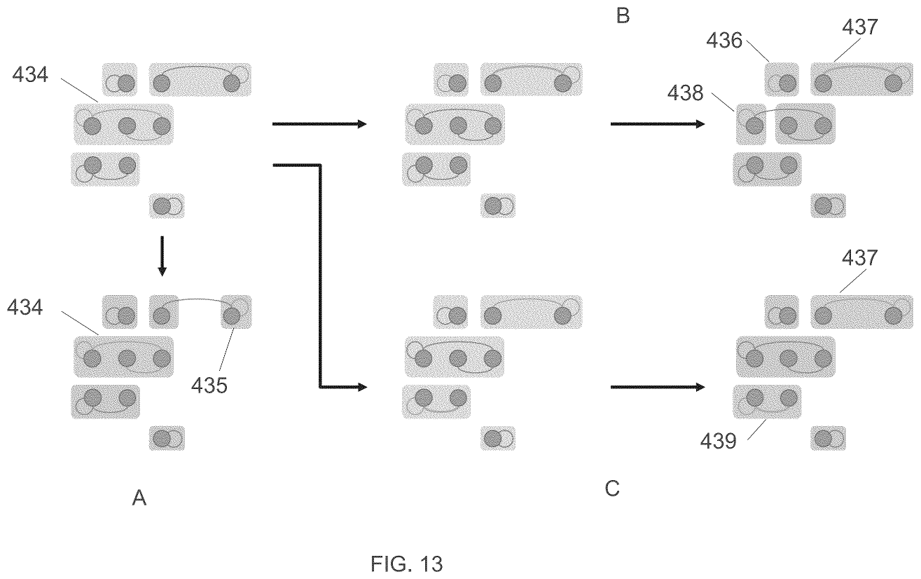

Next, the remaining conflicts are resolved using a scoring system. This process is illustrated by FIG. 13, which shows the process for the left-most sub-graph in FIG. 12. A similar process would be performed separately for the other sub-graph in FIG. 12. In the scoring process, the biggest of the possible clusters is firstly selected and

assumed to be correct (“enabled”). Thus, as shown in FIG. 13 at A, the cluster labelled 434 is firstly enabled. Any other clusters that conflict with this biggest cluster are then disabled. Next, the next biggest possible cluster (that has not yet been enabled or disabled) is enabled, and so on until a first explanation (or “assignment” or “arrangement”) is arrived at. Thus, in the example of FIG. 13 at A, the bottom image represents this initial explanation, in which the group of features-per-file are explained by two clusters 434, 435.

This explanation is then given an assignment score. To calculate the assignment score, each ion type assignment (loop) is given a relative weight factor. Any suitable weight factor(s) may be used. For example, a highest relative weight may be used for the most common adducts (e.g. [M+H] or [M−H]), a somewhat lower weight may be used for common adducts (e.g. [M+Na] or [M+K]), and an even lower weight may be used for the rest (e.g. uncommon adducts). The relative size of the various weight factor(s) may be configured as desired, e.g. to reflect the probability of a specific adduct appearing under the particular experimental conditions/sample chemistry used to obtain the data, and may be set by the user.

In addition, each transition is scored as twice the sum of its two ion weight factors. A factor of two is used here because a transition explains two nodes, while a loop assignment merely explains one node. Since the default assignment (loop) is typically the adduct with the highest weight, the algorithm would otherwise strongly prefer orphans assigned as the default adducts, which is not desired.

Then, each cluster is given a cluster score which is the sum of all loop and transition scores for the cluster, divided by the number of nodes in the cluster.

Finally, an assignment score is calculated for the explanation, which is the sum of all cluster scores for all enabled clusters in the explanation, divided by the number of clusters. The assignment score is designed to produce a high score for situations with lots of similar clusters are present (and to avoid, e.g., explanations containing just one large cluster and lots of orphans).

Returning again to FIG. 13, next, one or more other possible explanations are tested. As shown in FIG. 13 at B, to do this, one of the clusters that was disabled in the first step (i.e. FIG. 13 at A) is enabled, and the above process is repeated to produce a competing explanation. Thus, for example, in FIG. 13 at B, the right image represents this competing explanation, in which the group of features-per-file are now explained by three clusters 436, 437, 438. This explanation is again scored using the same scoring system.

Again, another possible explanation is tested by enabling a different one of the clusters disabled in the first step, and the above process repeated to produce another competing explanation. Thus, for example, in FIG. 13 at C, the right image represents this further competing explanation, in which the group of features-per-file are now explained by two clusters 437, 439. This explanation is again scored using the same scoring system.

This process may be repeated, e.g. until all possible explanations have been scored. Then, the explanation with the highest assignment score may be chosen as the final explanation for output.

Thus, the possible clusters are recursively evaluated to reach the best possible assignment score, where the score design reflects the nature of the analysed samples, e.g. in that under specific chromatographic conditions, most compounds should behave similarly and therefore create similar ion clusters.

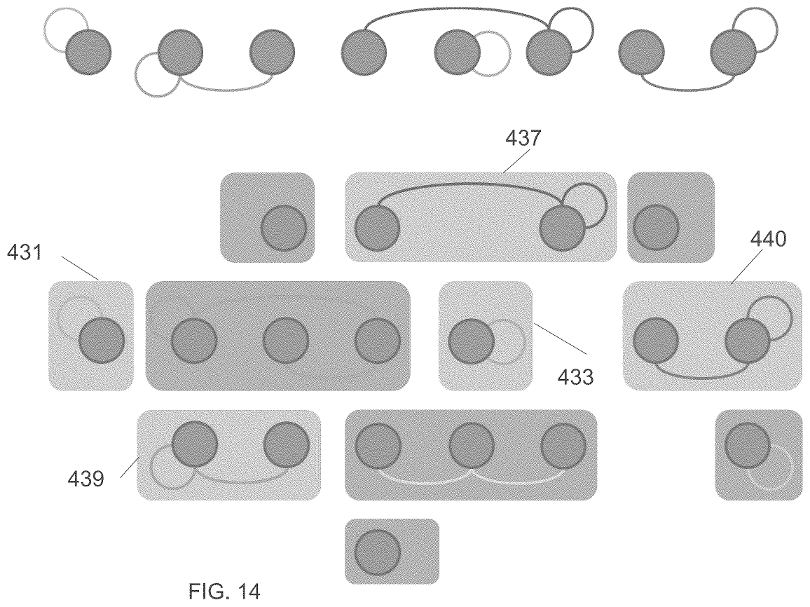

FIG. 14 shows an example final explanation, in which the group of features-per-file from FIG. 9 are explained by five clusters 431, 433, 437, 439, 440. This final explanation is added to the main graph 510.

Where there are multiple sample files, a similar process is performed for each sample file, and one explanation per sample file is added to the main graph 510 (and any duplicates are removed). This may result in the main graph 510 having some conflicting explanations (i.e. similarly to FIG. 12).

Thus, returning to FIG. 2, in step 500, any conflicting explanations in the main graph 510 are resolved. This process is performed using the same algorithm as is described above (with reference to FIGS. 12 to 14). This step ensures that the information from all sample files is taken into account, and that a consistent assignment across the files is achieved.

Each resulting sub-cluster represents a unique compound. Thus, finally, from each cluster in the final main graph, a compound can be identified (step 520 of FIG. 2).

Although various particular embodiments have been described above, various alternatives and additions are possible.

For example, one possible further step in the processing pipeline may be a quantitative analysis of the results. For this, it is necessary to know which features within a cluster can be used and which should be ignored (e.g. to sum up their areas for quantitation). Typically, any fragments are ignored and only ions assigned as a meaningful adduct are used for this. However, it is possible that a fragment should be used for quantitation, e.g. where it is related to a real adduction. For example, a common fragment of [M+NH4] is [M+H]. An additional step may therefore be applied to mark real adduct ions to be used for further processing, while any generic fragments should be ignored.

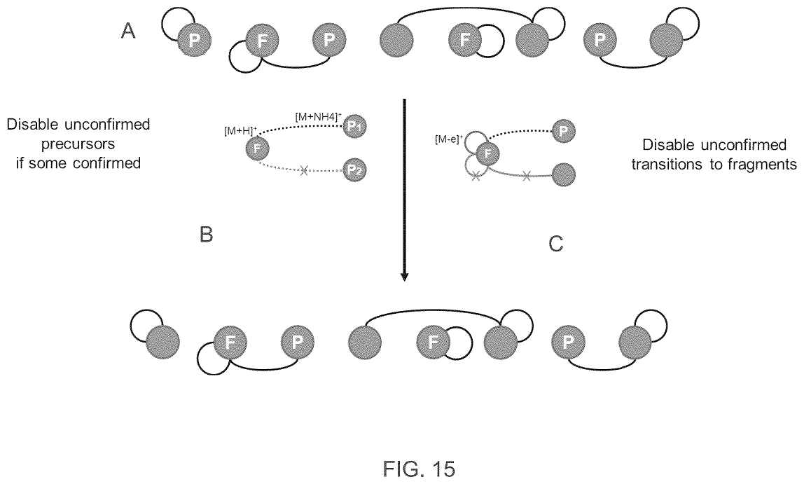

This process is illustrated by FIG. 15, where, as illustrated by FIG. 15 at A, it is possible that some fragments are shared by multiple precursors. Where this is the case, as illustrated by FIG. 15 at B, if the relationship to one of the precursors is also a valid adduct transition, then the relationship to any other precursor is rejected. This means that the fragment can be kept for further use, while avoiding using it multiple times. As illustrated by FIG. 15 at C, if for a fragment there are some other adduct transitions from features where the fragment-precursor relationship was not detected, those assignments are rejected, and the fragment is assigned as the generic fragment ion.

The methods described herein have general applicability to mass spectrometric analysis, e.g. of small molecules such as metabolic pathway analysis, degradation product analysis, forensic analysis, and so on.

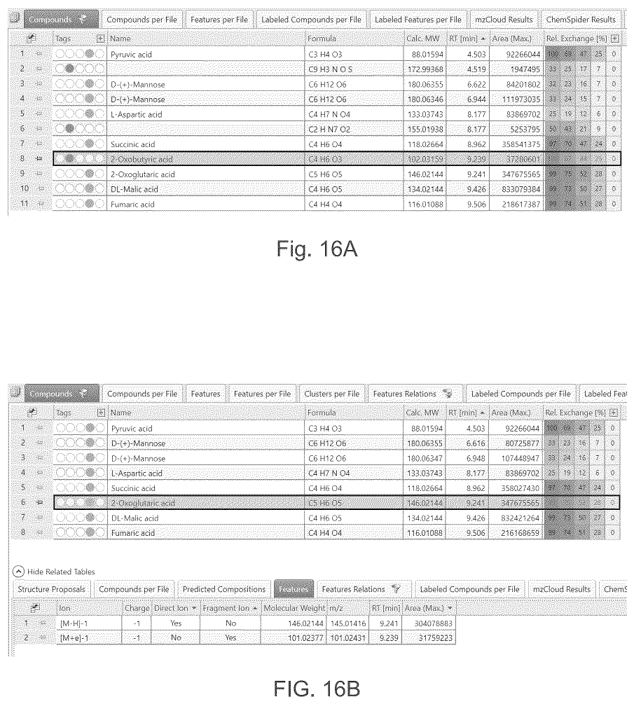

FIG. 16 shows example results from an artificial sample of eight compounds mixed with respective [13]C labelled analogues in known ratios. Due to the unique labelling, it was possible to determine the compounds together with all possible analogues and evaluate the method.

As shown in FIG. 16A, the conventional technique clearly reported three additional compounds showing the expected isotope exchange profile but having a close retention time to one of the expected compounds. Consideration of the assigned molecular formulae, and especially the formula difference between compounds at the same retention time, suggested an incorrect interpretation.

As shown in FIG. 16B, the same data were analysed by the method of compound assembly in accordance with embodiments. In this case, all three additional compounds were correctly interpreted as in-source fragments and were attached to their respective precursors.

The compound assembly method of various embodiments enables complete and consistent assembly of multiple diverse forms of ions originating from a single compound within multiple samples. Unlike the conventional techniques based exclusively on expected mass shifts within MS1 spectra, embodiments additionally utilise MS2 fragmentation spectra and on-line identification tools for initial adduct assignment, as well as for untargeted in-source fragments detection. The results from these steps are consolidated into unique ion clusters using a set of chemical and heuristic rules. This strategy significantly decreases the number of false positive identifications.

In accordance with embodiments, this is done, in particular, by:
(i) Applying a two-step grouping mechanism to consolidate possibly related features across plural different sample files. This enables consistent ion assignment across multiple different sample files.
(ii) Utilising acquired MS2 data to pre-identify compounds to provide high-confidence adduct assignment candidates based on spectral library matches. This avoids further ion misassignment of identifiable compounds.
(iii) Utilising curated low-energy collision spectra of already identified compounds in the data to then search for specific in-source fragment candidates. This helps to reduce the number of false positive unique compounds.
(iv) Utilising acquired MS2 spectra to search for matching in-source fragment candidates. This helps to reduce the number of false positive unique compounds in cases were low-energy collision spectra are not available.
(v) Applying a set of rules to the network of all possible ion relationships to remove assignments with low confidence. This reduces the chance of wrong adduct assignments.
(vi) Applying a custom scoring and evaluation mechanism to the consolidated network of ion relationships to resolve possible conflicting assignments. This creates individual ion clusters corresponding to unique compounds.

Although the present invention has been described with reference to various embodiments, it will be understood that various changes may be made without departing from the scope of the invention as set out in the accompanying claims.

1. A method of processing mass spectral data, the mass spectral data comprising a plurality of MS1 mass spectra and a plurality of MS^{N} (N≥2) mass spectra, with each mass spectrum having a respective associated retention time, the method comprising:
detecting a group of features in the plurality of MS1 mass spectra, wherein each feature of the group has a respective mass, and wherein the features of the group have corresponding retention times;
for each of one or more features of the group: (i) submitting a corresponding MS^{N} mass spectrum to a mass spectral search engine in order to obtain an identification result for that feature, and (ii) determining a candidate ion type for the feature based on a mass difference between the mass associated with the feature and an expected mass from the identification result; and then
identifying one or more compounds based on the group of features and the candidate ion type(s).

2. The method of claim 1, wherein the step of (ii) determining a candidate ion type for the feature comprises: determining a candidate adduction type for the feature based on the mass difference between the mass of the feature and the expected mass from the identification result.

3. The method of claim 1, wherein the mass spectral data comprises at least one sample file, with each sample file corresponding to a respective chromatographic separation scan and comprising plural MS¹ mass spectra and plural MS^{N} mass spectra, and wherein the step of detecting a group of features in the plurality of MS¹ mass spectra comprises:
for each sample file: detecting a plurality of features-per-file in that sample file, with each feature-per-file having a respective mass and a respective retention time;
forming a plurality of features from the features-per-file, with each feature having a respective mass and a respective retention time; and
forming a group of features by grouping features that have corresponding retention times.

4. The method of claim 3, wherein the step of detecting a plurality of features-per-file in a sample file comprises:
constructing a plurality of chromatograms from the plural MS¹ mass spectra of the sample file, with each chromatogram having a respective mass-to-charge ratio (m/z);
determining a characteristic retention time for each chromatogram;
grouping chromatograms that have corresponding characteristic retention times into one or more sets of chromatograms; and
applying a de-isotoping algorithm to each set of chromatograms to form a group of features-per-file.

5. The method of claim 4, wherein:
the step of grouping features that have corresponding retention times comprises grouping features that have retention times that are equal within a first tolerance;
the step of grouping chromatograms that have corresponding characteristic retention times comprises grouping chromatograms that have retention times that are equal within a second tolerance; and
the second tolerance is less than the first tolerance.

6. The method of claim 3, wherein the mass spectral data comprises a plurality of sample files, and each feature of the plurality of features is formed by grouping features-per-file that have corresponding masses and corresponding retention times.

7. The method of claim 6, wherein each group of features is formed from a corresponding group of features-per-file in each sample file, and wherein the step of identifying one or more compounds based on the group of features comprises:
for each group of features-per-file in a respective sample file:
(i) determining one or more clusters of features-per-file, wherein each cluster of features-per-file includes one or more features-per-file of the group, and possibly corresponds to a respective compound;
(ii) determining, for the group of features-per-file, one or more arrangements of the clusters of features-per-file, wherein each arrangement includes one or more non-conflicting clusters of features-per-file; and
(iii) selecting, for the group of features-per-file, a preferred arrangement from the one or more arrangements of clusters of features-per-file;
and then, based on the preferred arrangements for the plurality of sample files:
(iv) determining, for the group of features, one or more arrangements of clusters of features, wherein each cluster of features includes one or more features of the group of features and possibly corresponds to a respective compound, and wherein each arrangement includes one or more non-conflicting clusters of features; and
(v) selecting, for the group of features, a preferred arrangement from the one or more arrangements of clusters of features; and then
identifying one or more compounds based on the preferred arrangement of clusters of features.

8. A method of processing mass spectral data, the mass spectral data comprising a plurality of sample files, with each sample file comprising plural MS¹ mass spectra and plural MS^{N} (N≥2) mass spectra, with each mass spectrum having a respective associated retention time, the method comprising:
for each sample file: detecting a plurality of features-per-file in the MS¹ mass spectra of that sample file, with each feature-per-file having a respective mass and a respective retention time;
forming a plurality of features from the features-per-file by grouping features-per-file that have corresponding masses and corresponding retention times;
forming a group of features by grouping features that have corresponding retention times, and forming a corresponding group of features-per-file in each sample file; and then
for each group of features-per-file in a respective sample file:
(i) determining one or more clusters of features-per-file, wherein each cluster of features-per-file includes one or more features-per-file of the group, and possibly corresponds to a respective compound;
(ii) determining, for the group of features-per-file, one or more arrangements of the clusters of features-per-file, wherein each arrangement includes one or more non-conflicting clusters of features-per-file; and
(iii) selecting, for the group of features-per-file, a preferred arrangement from the one or more arrangements of clusters of features-per-file;
and then, based on the preferred arrangements for the plurality of sample files:
(iv) determining, for the group of features, one or more arrangements of clusters of features, wherein each cluster of features includes one or more features of the group of features and possibly corresponds to a respective compound, and wherein each arrangement includes one or more non-conflicting clusters of features; and
(v) selecting, for the group of features, a preferred arrangement from the one or more arrangements of clusters of features; and then
identifying one or more compounds based on the preferred arrangement of clusters of features.

9. The method of claim 7, wherein the step of determining one or more clusters of features-per-file comprises, for each group of features-per-file in a respective sample file:
assigning one or more candidate ion types to each feature-per-file of the group;
determining one or more candidate relationships between features-per-file of the group; and
resolving any conflicts between the candidate ion types and the candidate relationships.

10. The method of claim 9, wherein each feature-per-file has a respective charge, and wherein the step of assigning one or more candidate ion types to each feature-per-file of the group comprises:
assigning an identified ion type to any feature-per-file of the group that corresponds to a feature for which an identification result was obtained; and/or
assigning a user-defined base ion type or a default ion type to each feature-per-file of the group based on the respective charge of the feature-per-file.

11. The method of claim 9, wherein:
the step of assigning one or more candidate ion types to each feature-per-file of the group comprises: assigning an in-source fragment ion type to any feature-per-file of the group that has a mass corresponding to the mass of an expected in-source fragment of another feature-per-file in the group; and/or
the step of determining one or more candidate relationships between features-per-file of the group comprises: determining an in-source fragment relationship between a feature-per-file of the group and another feature-per-file of the group when the feature-per-file has a mass corresponding to the mass of an expected in-source fragment of the other feature-per-file.

12. The method of claim 11, further comprising obtaining the mass of an expected in-source fragment of a feature-per-file by:
determining the mass of an expected in-source fragment of a feature-per-file from an MS^{N} mass spectrum corresponding to the feature-per-file.

13. The method of claim 11, further comprising obtaining the mass of an expected in-source fragment of a feature-per-file by:
providing, as part of the identification result for a feature, the mass of one or more expected in-source fragments of the feature.

14. A method of processing mass spectral data, the mass spectral data comprising a plurality of MS¹ mass spectra and a plurality of MS^{N} (N≥2) mass spectra, with each mass spectrum having a respective associated retention time, the method comprising:
detecting a group of features in the plurality of MS¹ mass spectra, wherein each feature of the group has a respective mass, and wherein the features of the group have corresponding retention times;
for each of one or more features of the group: (i) submitting a corresponding MS^{N} mass spectrum to a mass spectral search engine in order to obtain an identification result for that feature, and (ii) providing, as part of the identification result, the mass of one or more expected in-source fragments of the feature;
assigning an in-source fragment ion type to any feature of the group that has a mass corresponding to the mass of an expected in-source fragment of another feature in the group; and
identifying one or more compounds based on the group of features and the in-source fragment ion type(s).

15. The method of claim 13, wherein the mass(es) provided as part of the identification result are determined from one or more MS^{N} mass spectra configured to simulate in-source fragmentation.

16. The method of claim 9, wherein the step of determining one or more candidate relationships between features-per-file of the group comprises:
determining one or more candidate adduct relationships between features-per-file of the group based on allowed mass shifts between features-per-file of the group.

17. The method of claim 7, wherein the step of selecting a preferred arrangement of clusters from the one or more arrangements of clusters comprises:
determining a score for each arrangement of clusters; and
selecting the arrangement with the highest score.

18. The method of claim 17, wherein the step of determining a score for each arrangement of clusters comprises:
determining a cluster score for each cluster in the arrangement by: (i) assigning a weight factor to each candidate ion type assignment of the cluster, (ii) assigning a relationship score to each candidate relationship of the cluster, and (iii) calculating a cluster score for the cluster by dividing the sum of the weight factors and relationship scores by the number of features or features-per-file in the cluster; and
determining a score for each arrangement by: dividing the sum of the cluster scores for the arrangement by the number of clusters in the arrangement.

19. A method of mass spectrometry comprising:
analysing a sample to obtain mass spectral data that comprises a plurality of MS¹ mass spectra and a plurality of MS^{N} mass spectra, with each mass spectrum having a respective associated retention time; and
processing the mass spectral data using the method of claim 1.

20. A non-transitory computer readable storage medium storing computer software code which when executed on a processor performs the method of claim 1.

21. A control system for an analytical instrument, the control system configured to cause the analytical instrument to perform the method of claim 1.

22. An analytical instrument comprising the control system of claim 21.
